(19) United States
(12) Patent Application Publication
(10) Pub. No.: US 2026/0179224 A1
(43) Pub. Date: Jun. 25, 2026

(54) IMAGE PROCESSING DEVICE, IMAGE PROCESSING METHOD, AND STORAGE MEDIUM

(71) Applicant: NEC Corporation, Tokyo (JP)

(72) Inventors: Kazuhiro WATANABE, Tokyo (JP); Yuji IWADATE, Tokyo (JP); Masahiro SAIKOU, Tokyo (JP); Akinori EBIHARA, Tokyo (JP); Taiki MIYAGAWA, Tokyo (JP)

(73) Assignee: NEC Corporation, Tokyo (JP)

(21) Appl. No.: 19/542,939

(22) Filed: Feb. 18, 2026

Related U.S. Application Data
(63) Continuation of application No. 18/561,130.

(30) Foreign Application Priority Data
Oct. 6, 2022 (WO) PCT/JP2022/037418

(51) Int. Cl. G06T 7/00; A61B 1/00; G06T 11/23; G06T 11/26; G06V 10/74; G16H 50/50

(52) U.S. Cl. CPC G06T 7/0012; A61B 1/000094; A61B 1/000096; A61B 1/00045; A61B 1/0005; G06T 11/23; G06T 11/26; G06V 10/761; G16H 50/50; A61B 1/000095; G06T 2207/10068; G06T 2207/20084; G06T 2207/30096; G06T 2210/41

(57) ABSTRACT

The image processing device 1X includes an acquisition means 30X and a lesion detection means 34X. The acquisition means 30X acquires an endoscopic image obtained by photographing an examination target by a photographing unit provided in an endoscope. The lesion detection means 34X detects a lesion based on a selection model which is selected from a first model and a second model, the first model being configured to make an inference regarding a lesion of the examination target based on a predetermined number of endoscopic images, the second model being configured to make an inference regarding a lesion of the examination target based on a variable number of endoscopic images. Besides, the lesion detection means 34X changes a parameter to be used for detection of the lesion based on a non-selection model that is the first model or the second model other than the selection model.

This application is a Continuation of U.S. application Ser. No. 18/561,130 filed Nov. 15, 2023, which is a National Stage of International Application No. PCT/JP2023/029842 filed Aug. 18, 2023, claiming priority based on International Application No. PCT/JP2022/037418 filed Oct. 6, 2022, the contents of all of which are incorporated herein by reference, in their entirety.

### TECHNICAL FIELD

The present disclosure relates to a technical field of an image processing device, an image processing method, and a storage medium for processing an image to be acquired in endoscopic examination.

### BACKGROUND

An endoscopic examination system for displaying images taken in the lumen of an organ is known. For example, Patent Literature 1 discloses a learning method of a learning model that outputs information relating to a lesion part included in an endoscope image data when the endoscope image data generated by a photographing device is inputted thereto. Further, Patent Literature 2 discloses a classification method for classifying series data through an application method of the sequential probability ratio test (SPRT: Sequential Probability Ratio Test). Non-Non-Patent Literature 1 also discloses an approximate computation method of the matrix when performing multi-class classification in the SPRT-based method disclosed in Patent Literature 2.

### CITATION LIST

### Patent Literature

Patent Literature 1: WO2020/003607
Patent Literature 1: WO2020/194497

### Non-Patent Literature

Non-Patent Literature 1: Miyagawa Taiki, and Akinori F. Ebihara. “The Power of Log-Sum-Exp: Sequential Density Ratio Matrix Estimation for Speed-Accuracy Optimization.” International Conference on Machine Learning. PMLR, 2021.

### SUMMARY

### Problem to be Solved

In the case of detecting a lesion from images taken in the endoscopic examination, there are a method of detecting the lesion based on a fixed predetermined number of images, and a method of detecting the lesion based on a variable number of images as described in Patent Literature 2. Then, the lesion detection method based on a fixed number of images leads to lesion detection with high accuracy even when there is no change in the images, but there is an issue that it is susceptible to noise such as blurring. In contrast, the lesion detection method based on a variable number of images described in Patent Literature 2 is less susceptible to instantaneous noises while being able to easily detect a distinguishable lesion, however, there is an issue that it could lead to detection delay or miss of the lesion when there is no substantial change between the images.

In view of the above-described issue, it is therefore an example object of the present disclosure to provide an image processing device, an image processing method, and a storage medium capable of suitably detecting a lesion in an endoscopic image.

### Means for Solving the Problem

One mode of the image processing device is an image processing device including:
an acquisition means configured to acquire an endoscopic image obtained by photographing an examination target by a photographing unit provided in an endoscope; and
a lesion detection means configured to detect a lesion based on a selection model which is selected from a first model and a second model,
   the first model being configured to make an inference regarding a lesion of the examination target based on a predetermined number of endoscopic images,
   the second model being configured to make an inference regarding a lesion of the examination target based on a variable number of endoscopic images,
wherein the lesion detection means is configured to change a parameter to be used for detection of the lesion based on a non-selection model that is the first model or the second model other than the selection model.

One mode of the image processing method is an image processing method executed by a computer, the image processing method including:
acquiring an endoscopic image obtained by photographing an examination target by a photographing unit provided in an endoscope;
detecting a lesion based on a selection model which is selected from a first model and a second model,
   the first model being configured to make an inference regarding a lesion of the examination target based on a predetermined number of endoscopic images,
   the second model being configured to make an inference regarding a lesion of the examination target based on a variable number of endoscopic images; and
changing a parameter to be used for detection of the lesion based on a non-selection model that is the first model or the second model other than the selection model.

One mode of the storage medium is a storage medium storing a program executed by a computer, the program causing the computer to:
acquire an endoscopic image obtained by photographing an examination target by a photographing unit provided in an endoscope;
detect a lesion based on a selection model which is selected from a first model and a second model,
   the first model being configured to make an inference regarding a lesion of the examination target based on a predetermined number of endoscopic images,
   the second model being configured to make an inference regarding a lesion of the examination target based on a variable number of endoscopic images; and
change a parameter to be used for detection of the lesion based on a non-selection model that is the first model or the second model other than the selection model.

### Effect

An example advantage according to the present disclosure is to suitably detect a lesion in an endoscope image.

### BRIEF DESCRIPTION OF THE DRAWINGS

FIG. 1 It illustrates a schematic configuration of an endoscopic examination system.

FIG. 2 It illustrates a hardware configuration of an image processing device.

FIG. 3 It is a functional block diagram of the image processing device.

FIG. 4 It illustrates an example of a display screen image displayed by a display device in endoscopic examination.

FIG. 5A is a graph according to a first specific example showing the transition of the first score from the processing time t0 at which the acquisition of endoscopic images was started.

FIG. 5B is a graph according to the first specific example showing the transition of the second score from the processing time t0.

FIG. 6A is a graph according to a second specific example showing the transition of the first score from the processing time t0.

FIG. 6B is a graph according to the second specific example showing the transition of the second score from the processing time t0.

FIG. 7 It is an example of a flowchart that the image processing device executes in the first example embodiment.

FIG. 8A is a graph according to a second example embodiment showing the transition of the first score from the processing time t0.

FIG. 8B is a graph according to the second example embodiment showing the transition of the second score from the processing time t0.

FIG. 9 It is an example of a flowchart executed by the image processing device in the second example embodiment.

FIG. 10 It is an example of a flowchart executed by the image processing device in a third example embodiment.

FIG. 11 It is a block diagram of an image processing device according to a fourth example embodiment.

FIG. 12 It is an example of a flowchart executed by the image processing device in the fourth example embodiment.

### EXAMPLE EMBODIMENTS

Hereinafter, example embodiments of an image processing device, an image processing method, and a storage medium will be described with reference to the drawings.

### First Example Embodiment

### (1-1) System Configuration

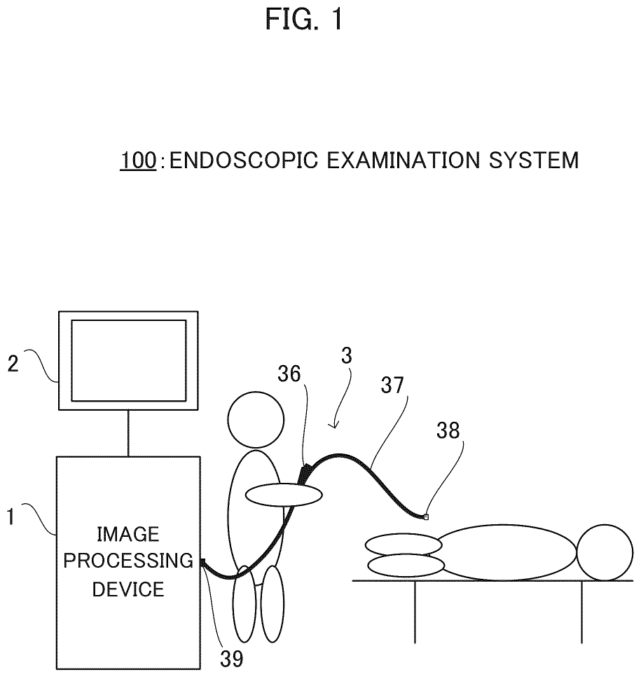

FIG. 1 shows a schematic configuration of an endoscopic examination system 100. The endoscopic examination system 100 detects a part (a lesion part) of examination target which is suspected of a lesion and presents the detection result to an examiner such as a doctor who performs examination or treatment using an endoscope. This allows the endoscopic examination system 100 to assist in decision making, such as the determination of a treatment policy for the subject of the examination, by the examiner such as a doctor. As shown in FIG. 1, the endoscopic examination system 100 mainly includes an image processing device 1, a display device 2, and an endoscope 3 connected to the image processing device 1.

The image processing device 1 acquires an image (also referred to as “endoscopic image Ia”) captured by the endoscope 3 in time series from the endoscope 3 and displays a screen image based on the endoscopic image Ia on the display device 2. The endoscopic image Ia is an image captured at predetermined time intervals in at least one of the insertion process of the endoscope 3 to the subject or the ejection process of the endoscope 3 from the subject. In the present example embodiment, the image processing device 1 analyzes the endoscopic image Ia to detect the endoscopic image Ia in which the lesion part is included, and displays the information regarding the detection result on the display device 2.

The display device 2 is a display or the like for display information based on the display signal supplied from the image processing device 1.

The endoscope 3 mainly includes an operation unit 36 for examiner to perform a predetermined input, a shaft 37 which has flexibility and which is inserted into the organ to be photographed of the subject, a pointed end unit 38 having a built-in photographing unit such as an ultra-small image pickup device, and a connecting unit 39 for connecting with the image processing device 1.

The configuration of the endoscopic examination system 100 shown in FIG. 1 is an example, and various change may be applied thereto. For example, the image processing device 1 may be configured integrally with the display device 2. In another example, the image processing device 1 may be configured by a plurality of devices.

Hereafter, as a representative example, the description will be given of the process in the endoscopic examination of the large bowel. However, the examination target is not limited to the large bowel and it may be an esophagus or stomach. Examples of the target of the endoscopic examination in the present disclosure include a laryngendoscope, a bronchoscope, an upper digestive tube endoscope, a duodenum endoscope, a small bowel endoscope, a large bowel endoscope, a capsule endoscope, a thoracoscope, a laparoscope, a cystoscope, a cholangioscope, an arthroscope, a spinal endoscope, a blood vessel endoscope, and an epidural endoscope. In addition, the conditions of the lesion part to be detected in endoscopic examination are exemplified as (a) to (f) below.
(a) Head and neck: pharyngeal cancer, malignant lymphoma, papilloma
(b) Esophagus: esophageal cancer, esophagitis, esophageal hiatal hernia, Barrett's esophagus, esophageal varices, esophageal achalasia, esophageal submucosal tumor, esophageal benign tumor
(c) Stomach: gastric cancer, gastritis, gastric ulcer, gastric polyp, gastric tumor
(d) Duodenum: duodenal cancer, duodenal ulcer, duodenitis, duodenal tumor, duodenal lymphoma
(e) Small bowel: small bowel cancer, small bowel neoplastic disease, small bowel inflammatory disease, small bowel vascular disease
(f) Large bowel: colorectal cancer, colorectal neoplastic disease, colorectal inflammatory disease; colorectal polyps, colorectal polyposis, Crohn's disease, colitis, intestinal tuberculosis, hemorrhoids.

### (1-2) Hardware Configuration

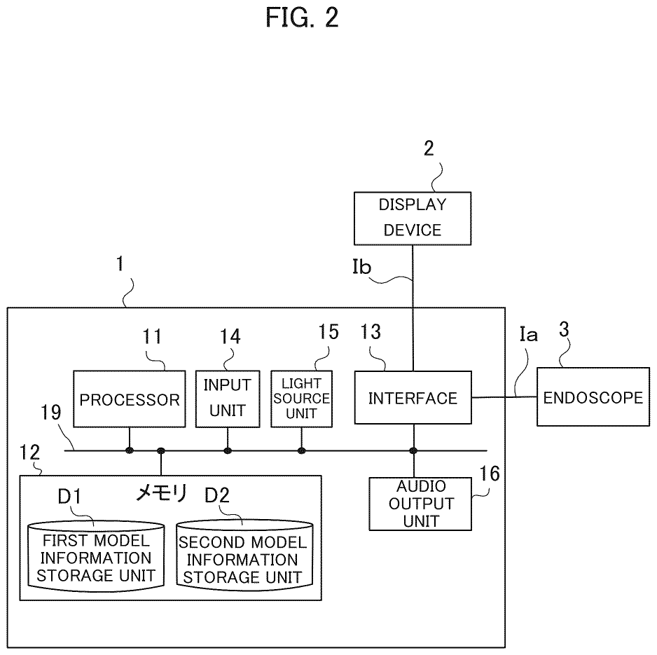

FIG. 2 shows the hardware configuration of the image processing device 1. The image processing device 1 mainly includes a processor 11, a memory 12, an interface 13, an input unit 14, a light source unit 15, and an audio output unit 16. Each of these elements is connected via a data bus 19.

The processor 11 executes a predetermined process by executing a program or the like stored in the memory 12. The processor 11 is one or more processors such as a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by a variety of volatile memories which are used as working memories, and nonvolatile memories which store information necessary for the process to be executed by the image processing device 1, such as a RAM (Random Access Memory) and a ROM (Read Only Memory). The memory 12 may include an external storage device such as a hard disk connected to or built in to the image processing device 1, or may include a storage medium such as a removable flash memory. The memory 12 stores a program for the image processing device 1 to execute each process in the present example embodiment.

Further, the memory 12 functionally includes a first model information storage unit D1 for storing first model information, and a second model information storage unit D2 for storing second model information. The first model information includes parameter information of a first model that is used by the image processing device 1 to detect a lesion part. The first model information may further include information indicating the calculation result of the detection process of the lesion part using the first model. The second model information includes parameter information of a second model that is used by the image processing device 1 to detect a lesion part. The second model information may further include information indicating the calculation result of the detection process of the lesion part using the second model.

The first model is a model to make an inference regarding a lesion part of the examination target based on a fixed predetermined number (may be one or may be multiple) of endoscopic images. Specifically, the first model is a model which learned the relation between a fixed predetermined number of endoscopic images or the feature values thereof, to be inputted to the first model, and a determination result regarding the lesion part in the endoscopic images. In other words, the first model is a model that is trained to output, when input data that is a fixed predetermined number of endoscopic images or their feature values is inputted thereto, a determination result regarding a lesion part in the endoscopic images. In the present example embodiment, the determination result regarding the lesion part outputted from the first model includes at least a score (an index value) regarding the presence or absence of the lesion part in the endoscopic images, and this score is hereafter also referred to as “first score S1”. For convenience of explanation, the first score S1 shall indicate that the higher the first score S1 is, the higher the degree of confidence that there is a lesion part in the endoscopic images of interest becomes. The above-described determination result regarding the lesion part may further include information indicating the position or region (area) of the lesion part in the endoscopic image.

The first model is, for example, a deep learning model which includes a convolutional neural network in its architecture. Examples of the first model include Fully Convolutional Network, SegNet, U-Net, V-Net, Feature Pyramid Network, Mask R-CNN, and DeepLab. The first model data storage unit D1 includes various parameters required for building the first model, such as a layer structure, a neuron structure of each layer, the number of filters and the size of filters in each layer, and the weight for each element of each filter. The first model is trained in advance on the basis of sets of: endoscopic images that are input data conforming to the input format of the first model, or feature values thereof; and correct answer data indicating a determination result of a correct answer regarding the lesion part in the endoscopic images.

The second model is a model configured to make an inference regarding a lesion part of the examination target based on a variable number of endoscopic images. Specifically, the second model is a model which learned, through machine learning, a relation between a variable number of endoscopic images or their feature values and a determination result regarding the lesion part in the endoscopic images. In other words, the second model is a model that is trained to output, when input data that is a variable number of endoscopic images or their feature values is inputted thereto, the determination result on the lesion part in the endoscopic images. In the present example embodiment, the “determination result regarding the lesion part” includes at least a score regarding the presence or absence of the lesion part in the endoscopic images, and this score is hereinafter also referred to as a “second score S2”. For convenience of explanation, the second score S2 shall indicate that the higher the second score S2 is, the higher the degree of confidence that there is a lesion part in the endoscopic images of interest becomes. Examples of the second model include a model based on SPRT described in Patent Literature 2. A specific example of the second model based on SPRT will be described later. Various parameters required for building the second model are stored in the second model information storage unit D2.

Further, in the memory 12, in addition to the first model information and the second model information, various information such as parameters necessary for the lesion detection process is stored. At least a portion of the information stored in the memory 12 may be stored in an external device other than the image processing device 1 instead. In this case, the above-described external device may be one or more server devices capable of performing data communication with the image processing device 1 through a communication network or through direct communication.

The interface 13 performs an interface operation between the image processing device 1 and an external device. For example, the interface 13 supplies the display information “Ib” generated by the processor 11 to the display device 2. Further, the interface 13 supplies the light generated by the light source unit 15 to the endoscope 3. The interface 13 also provides an electrical signal to the processor 11 indicative of the endoscopic image Ia supplied from the endoscope 3. The interface 13 may be a communication interface, such as a network adapter, for wired or wireless communication with the external device, or a hardware interface compliant with a USB (Universal Serial Bus), a SATA (Serial AT Attachment), or the like.

The input unit 14 generates an input signal based on the operation by the examiner. Examples of the input unit 14 include a button, a touch panel, a remote controller, and a voice input device. The light source unit 15 generates light for supplying to the pointed end unit 38 of the endoscope 3. The light source unit 15 may also incorporate a pump or the like for delivering water and air to be supplied to the endoscope 3. The audio output unit 16 outputs a sound under the control of the processor 11.

### (1-3) Outline of Lesion Detection Process

Next, an outline of the detection process (lesion detection process) of the lesion part by the image processing device 1 will be described. In summary, in the lesion detection based on the first score S1 outputted by the first model, the image processing device 1 changes a parameter to be used for the above-mentioned lesion detection, based on the second score S2 outputted by the second model. Specifically, the above-described parameter is such a parameter that defines a condition (criterion) for determining that a lesion is detected on the basis of the first score S1, and the image processing device 1 changes the parameter so that the higher the degree of confidence regarding existence of the lesion indicated by the second score S2 is, the more the above-described condition is relaxed. Thus, the image processing device 1 performs accurate lesion detection utilizing the advantages of both the first model and the second model, and presents the detection result. In the first example embodiment, the first model is an example of the “selection model” and the second model is an example of the “non-selection model”.

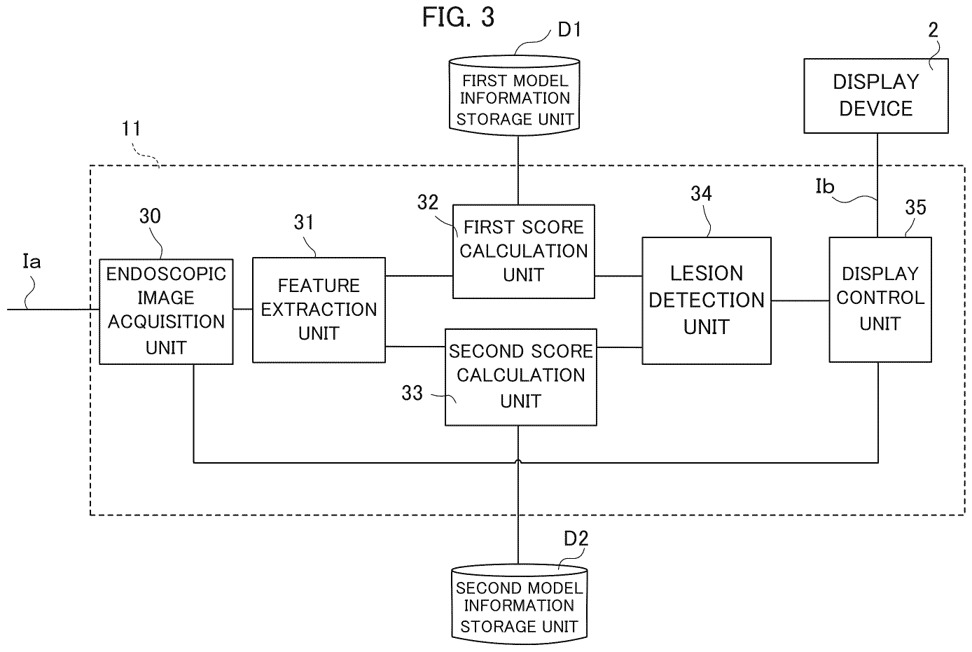

FIG. 3 is a functional block diagram of the image processing device 1. As shown in FIG. 3, the processor 11 of the image processing device 1 functionally includes an endoscopic image acquisition unit 30, a feature extraction unit 31, a first score calculation unit 32, a second score calculation unit 33, a lesion detection unit 34, and a display control unit 35. In FIG. 3, blocks to exchange data with each other are connected by a solid line, but the combination of blocks to exchange data with each other is not limited to the combination shown in FIG. 3. The same applies to the drawings of other functional blocks described below.

The endoscopic image acquisition unit 30 acquires the endoscopic image Ia captured by the endoscope 3 through the interface 13 at predetermined intervals according to the frame period of the endoscope 3, and supplies the acquired endoscopic image Ia to the feature extraction unit 31 and the display control unit 35. Then, each processing unit provided in the subsequent stage periodically performs the processing described later at the time intervals at which the endoscope image acquisition unit 30 acquires each endoscope image. Hereinafter, the time at the intervals of frame period is also referred to as “processing time”.

The feature extraction unit 31 converts the endoscopic image Ia supplied from the endoscopic image acquisition unit 30 into feature values (in details, feature vectors or tensor data in the third or more higher order) represented in a predetermined dimensional feature space. In this case, for example, the feature extraction unit 31 builds a feature extractor based on parameters stored in advance in the memory 12 or the like, and acquires feature values outputted by the feature extractor by inputting an endoscopic image Ia to the feature extractor. Here, the feature extractor may be a deep learning model with an architecture such as a convolutional neural network. In this case, machine learning is applied to the feature extractor in advance, and the parameters obtained through the machine learning are stored in advance in the memory 12 or the like. The feature extractor may extract feature values representing the relation among time series data, based on any technique for calculating the relation among the time series data such as LSTM (Long Short Term Memory). Then, the feature extraction unit 31 supplies the feature data representing the generated feature values to the first score calculation unit 32 and the second score calculation unit 33.

The above-described feature extractor may be incorporated in at least one of the first model and/or the second model. For example, when the architecture of the feature extractor is included in the first model, the first score calculation unit 32 inputs the endoscopic image Ia to the first model, and then supplies the feature data indicating the feature values generated by the feature extractor in the first model to the second score calculation unit 33 as the output from an intermediate layer of the first model. In this case, the feature extraction unit 31 may not be provided.

The first score calculation unit 32 calculates the first score S1 based on information stored in the first model information storage unit D1 and the feature data supplied from the feature extraction unit 31. In this instance, the first score calculation unit 32 acquires the first score S1 outputted by the first model by inputting the feature data supplied from the feature extraction unit 31 to the first model which is configured by referring to the first model information storage unit D1. If the first model is a model configured to output the first score S1 based on a single endoscopic image Ia, the first score calculation unit 32 calculates the first score S1 at the current processing time by inputting the feature data supplied at the current processing time from the feature extraction unit 31 to the first model, for example. In contrast, if the first model is a model configured to output the first score S1 on the basis of a plurality of endoscopic images Ia, the first score calculation unit 32 may calculate the first score S1 at the current processing time by inputting, into the first model, a combination of the feature data supplied from the feature extraction unit 31 at the current processing time and the feature data supplied in the past, for example. The first score calculation unit 32 may calculate the first score S1 (i.e., moving-averaged score) obtained by averaging the score(s) obtained at the past processing time(s) and the score obtained at the current processing time. The first score calculation unit 32 supplies the calculated first score S1 to the lesion detection unit 34.

The second score calculation unit 33 calculates the second score S2 indicating the likelihood of the presence of the lesion part based on information stored in the second model information storage unit D2 and the feature data corresponding to the variable number of time-series endoscopic images Ia obtained up to the present. In this instance, for each processing time, the second score calculation unit 33 determines the second score S2 based on the likelihood ratio regarding the time series endoscopic images Ia, which is calculated using the second model based on SPRT. Here, “likelihood ratio regarding the time series endoscopic images Ia” refers to the ratio of the likelihood that there is a lesion part in the time series endoscopic images Ia to the likelihood that there is no lesion part in the time series endoscopic images Ia. In the present example embodiment, as an example, it is assumed that the likelihood ratio increases with an increase in the likelihood of the presence of the lesion part. Specific examples of the method for calculating the second score S2 using the second model based on SPRT will be described later. The second score calculation unit 33 supplies the calculated second score S2 to the lesion detection unit 34.

The lesion detection unit 34 performs the lesion detection (i.e., determination of the presence or absence of the lesion part) in the endoscopic images Ia, based on the first score S1 supplied from the first score calculation unit 32 and the second score S2 supplied from the second score calculation unit 33. In this instance, the lesion detection unit 34 changes, based on the second score S2, a threshold value that defines a criterion for determining, based on the first score S1, that a lesion is detected. Specific examples of the process of the lesion detection unit 34 will be described later. The lesion detection unit 34 supplies the lesion detection result to the display control unit 35.

The display control unit 35 generates display information Ib, based on the endoscopic image Ia and the lesion detection result supplied from the lesion detection unit 34, then supplies the display information Ib to the display device 2 through the interface 13 to thereby cause the display device 2 to display information regarding the endoscopic image Ia and the lesion detection result outputted by the lesion detection unit 34. The display control unit 35 may further display information regarding the first score S1 calculated by the first score calculation unit 32 and the second score S2 calculated by the second score calculation unit 33 on the display device 2.

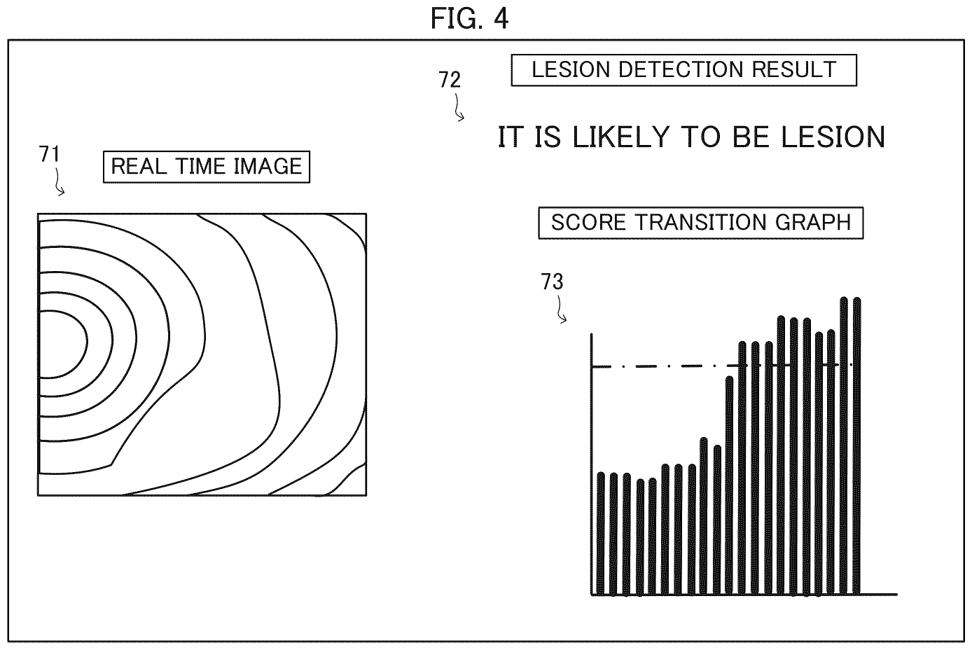

FIG. 4 shows an example of a display screen image displayed by the display device 2 in the endoscopic examination. The display control unit 35 of the image processing device 1 outputs display information Ib generated on the basis of the endoscopic image Ia acquired by the endoscopic image acquisition unit 30 and the lesion detection result by the lesion detection unit 34 by the display device 2. The display control unit 35 transmits the endoscopic image Ia and the display information Ib to the display device 2 to thereby display the above-described display screen image on the display device 2. In the example of the display screen image shown in FIG. 4, the display control unit 35 of the image processing device 1 provides a real time image display area 71, a lesion detection result display area 72, and a score transition display area 73 on the display screen image.

The display control unit 35 herein displays, in the real time image display area 71, a moving image representing the latest endoscopic image Ia. Furthermore, in the lesion detection result display area 72, the display control unit 35 displays the lesion detection result outputted by the lesion detection unit 34. At the time of providing the display screen image shown in FIG. 4, since the lesion part is determined to be present by the lesion detection unit 34, the display control unit 35 displays a text message indicating that there is likely to be a lesion, in the lesion detection result display area 72. Instead of displaying the text message indicating that there is likely to be a lesion in the lesion detection result display area 72, or in addition to this, the display control unit 35 may output a sound (including voice) notifying the user that it is likely to be a lesion, by the audio output unit 16.

Further, in the score transition display area 73, the display control unit 35 displays the score transition graph indicating the transition of the first score S1 from the start point of the endoscopic examination to the current point, together with a dashed line indicating the criterion value (first score threshold value Sth1 to be described later) for determining the presence or absence of a lesion by using the first score S1.

Each component of the endoscopic image acquisition unit 30, the feature extraction unit 31, the first score calculation unit 32, the second score calculation unit 33, the lesion detection unit 34 and the display control unit 35 can be realized, for example, by the processor 11 which executes a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

### (1-4) Example of Calculating Second Score

Next, an exemplary calculation of the second score S2 using the second model based on SPRT will be described.

The second score calculation unit 33 calculates the likelihood ratio relating to latest “N” number of endoscopic images Ia (N is an integer of 2 or more) for each processing time, and determines the second score S2 based on the likelihood ratio (also referred to as “integrated likelihood ratio”) into which the likelihood ratio calculated at the current processing time and the likelihood(s) at past processing time(s) are integrated. The second score S2 may be the integrated likelihood ratio itself or may be a function value including the integrated likelihood ratio as an argument. Hereinafter, for convenience of explanation, the second model shall include a likelihood ratio calculation model that is a processing unit for calculating the likelihood ratio and a score calculation model that is a processing unit for calculating the second score S2 from the likelihood ratio.

The likelihood ratio calculation model is a model that is trained to output, when feature data of N endoscopic images Ia is inputted thereto, the likelihood ratio regarding the N endoscopic images Ia. The likelihood ratio calculation model may be a deep learning model, a statistical model, or any other machine learning model. In this instance, for example, the learned parameters of the second model including the likelihood ratio calculation model are stored in the second model information storage unit D2. When the likelihood ratio calculation model is constituted by a neural network, various parameters such as a layer structure, a neuron structure of each layer, the number of filters and the filter size in each layer, and the weight for each element of each filter are stored in advance in the second model information storage unit D2. It is noted that even when the number of acquired endoscopic images Ia is less than N, the second score calculation unit 33 can acquire the likelihood ratio from the acquired less than N endoscopic images Ia using the likelihood ratio calculation model. The second score calculation unit 33 may store the acquired likelihood ratio in the second model information storage unit D2.

Next, the score calculation model included in the second model will be described. It is hereinafter assumed that the index “1” denotes a predetermined start time, the index “t” denotes the current processing time, and that “xᵢ” (i=1, . . . , t) denotes the feature values of the endoscopic images Ia to be processed at the processing time i. The “start time” represents the first processing time of the past processing times to be considered in the calculation of the second score S2. In this instance, the integrated likelihood ratio for the binary classification between the class “C₁” indicating that the endoscopic images Ia contain the lesion part and the class “C₀” indicating that the endoscopic images Ia does not contain the lesion part is expressed by the following equation (1).

$$log [ \frac{p ( {x}_{1} , … , {x}_{t} \vert {C}_{1} )}{p ( {x}_{1} , … , {x}_{t} \vert {C}_{0} )} ] = {\sum }_{s = N + 1}^{t} log [ \frac{p ( {C}_{1} \vert {x}_{s} , … , {x}_{s - N} )}{p ( {C}_{0} \vert {x}_{s} , … , {x}_{s - N} )} ] - {\sum }_{s = N + 2}^{t} log [ \frac{p ( {C}_{1} \vert {x}_{s - 1} , … , {x}_{s - N} )}{p ( {C}_{0} \vert {x}_{s - 1} , … , {x}_{s - N} )} ] \qquad [ Equation 1 ]$$

Here, “p” represents the probability (i.e., confidence score ranging from 0 to 1) belonging to each class. In calculating the term on the right-hand side of the equation (1), the likelihood ratio outputted by the likelihood ratio calculation model can be used.

In the equation (1), since the time index t representing the current processing time increases over time, the length (i.e., the number of frames) of the time series endoscopic images Ia used for calculating the integrated likelihood ratio is a variable length. Thus, the first advantage of using the integrated likelihood ratio based on the equation (1) is that the second score calculation unit 33 can calculate the second score S2 considering a variable number of the endoscopic images Ia. The second advantage of using the integrated likelihood ratio based on the equation (1) is that the time-dependent features can be classified. The third advantage thereof is that it is possible to calculate the second score S2 with sufficient accuracy even for discriminant-difficult data. The second score calculation unit 33 may store the integrated likelihood ratio and the second score S2 calculated at the respective processing times in the second model information storage unit D2.

The second score calculation unit 33 may determines that there is no lesion part if the second score S2 reaches a predetermined threshold value which is a negative value. In this case, the second score calculation unit 33 initializes the second score S2 and the time index t to 0, and restarts the calculation of the second score S2 on the basis of the endoscopic images Ia obtained at subsequent processing times.

### (1-5) Process by Lesion Detection Unit

Next, a description will be given of a specific determination method of the presence or absence of the lesion part by the lesion detection unit 34. For each processing time, the lesion detection unit 34 compares the first score S1 with a threshold value (also referred to as “first score threshold value Sth1”) for the first score S1 and compares the second score S2 with a threshold value (also referred to as “second score threshold value Sth2”) for the second score S2. Then, if the first score S1 consecutively exceeds the first score threshold value Sth1 for more than a predetermined times (also referred to as “threshold count Mth”), the lesion detection unit 34 determines that there is a lesion part. On the other hand, if the second score S2 becomes larger than the second score threshold value Sth2, the lesion detection unit 34 decreases the threshold count Mth. In this way, in such a situation where the presence of a lesion part is suspected based on the second score S2 outputted by the second model, the lesion detection unit 34 relaxes the condition for determining, based on the first score S1, that there is a lesion part. Thus, in each of the situation in which the first model is easy to accurately detect a lesion part and the situation in which the second model is easy to accurately detect a lesion part, it is possible to accurately detect the lesion part.

Hereafter, the number of times the first score S1 has consecutively exceeded the first score threshold value Sth1 is referred to as “over-threshold consecutive count M”. It is noted that fitting values for the first score threshold value Sth1 and the second score threshold value Sth2 are stored in advance in the memory 12 or the like, respectively, for example. The threshold count Mth is a value that varies according to the second score S2 and the initial value thereof is stored in advance in the memory 12 or the like. The threshold count Mth is an example of the “parameter to be used for detection of a lesion based on a selection model”.

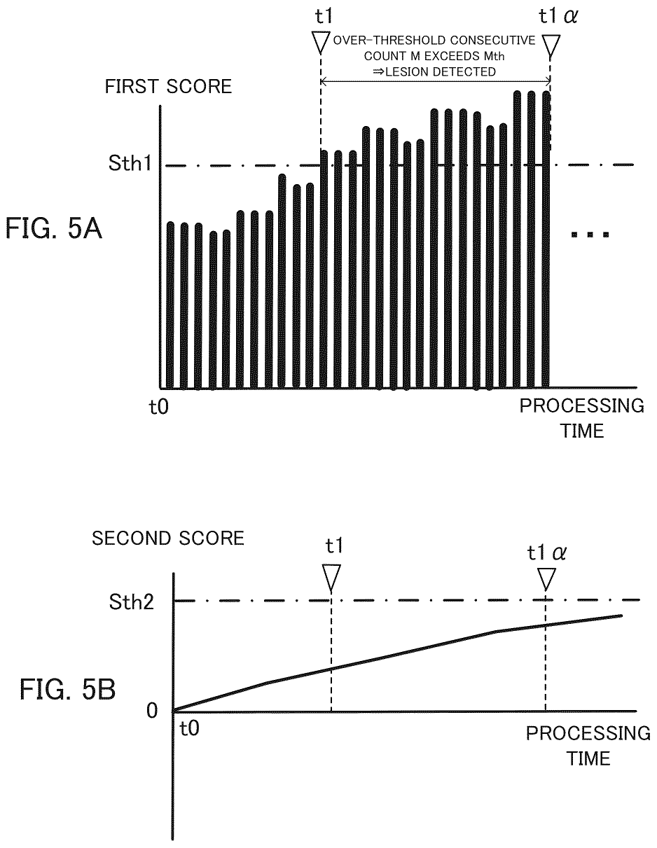

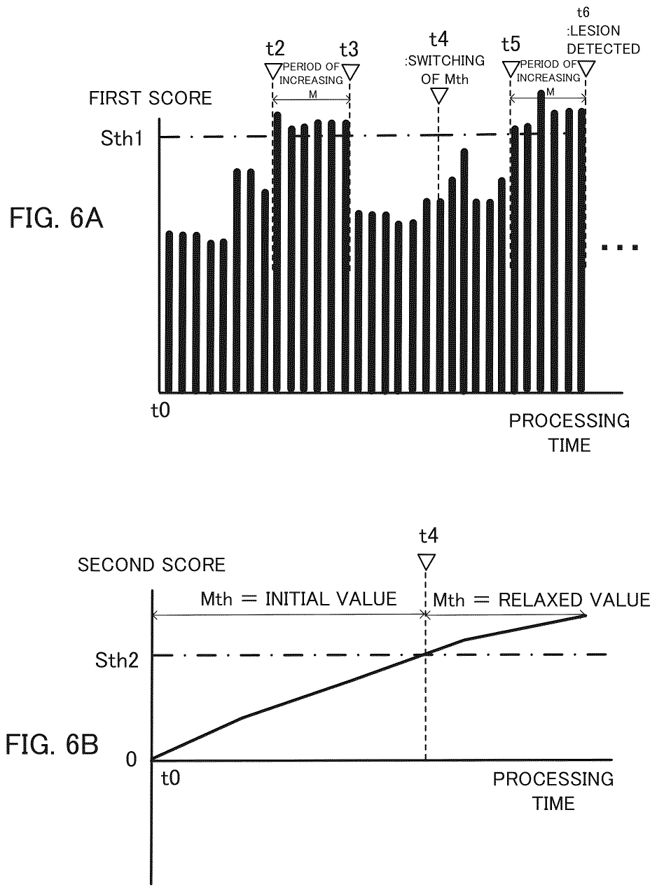

Next, a description will be given of a method of determining the lesion detection by the lesion detection unit 34 using a first specific example shown in FIGS. 5A and 5B, and a second specific example shown in FIGS. 6A and 6B.

FIG. 5A is a graph showing the transition of the first score S1 from the processing time “t0” at which acquisition of endoscope image Ia was started in the first specific example, and FIG. 5B is a graph showing the transition of the second score S2 from the processing time t0 in the first specific example. It is noted that the first specific example is an example of the lesion detection process in a situation where the accuracy of the lesion detection based on the first model becomes higher than the accuracy of the lesion detection based on the second model. Examples of such situation include a situation where the variation in endoscopic images Ia in time series is relatively small.

In the first specific example, at each processing time after the processing time t0, the lesion detection unit 34 compares the first score S1 with the first score threshold value Sth1 and compares the second score S2 with the second score threshold value Sth2, wherein the first score S1 and the second score S2 are obtained at each processing time, respectively. Then, at the processing time “t1”, the lesion detection unit 34 determines that the first score S1 exceeds the first score threshold value Sth1, and then starts counting the over-threshold consecutive count M. At the processing time “t1α”, the lesion detection unit 34 determines that the over-threshold consecutive count M has exceeded the threshold count Mth. Therefore, in this instance, the lesion detection unit 34 determines that there is a lesion part in the endoscopic images Ia obtained at the processing times t1 to t1α. On the other hand, after the processing time t0, the lesion detection unit 34 determines that the second score S2 is equal to or less than the second score threshold value Sth2, and fixes the threshold count Mth even after the processing time t0.

Thus, in such a situation in which the accuracy of the lesion detection based on the first model becomes higher than the accuracy of the lesion detection based on the second model, although the second score S2 based on the second model does not reach the second score threshold value Sth2, the first score S1 based on the first model stably reaches the first score threshold value Sth1. Therefore, in such a situation, the lesion detection unit 34 can perform the lesion detection accurately.

FIG. 6A is a graph showing the transition of the first score S1 from the processing time t0 in the second specific example, FIG. 6B is a graph showing the transition of the second score S2 from the processing time t0 in the second specific example. The second specific example is an example of the lesion detection process in such a situation in which the accuracy of the lesion detection based on the second model becomes higher than the accuracy of the lesion detection based on the first model. Examples of such situations include a situation where the variation in endoscopic images Ia in time series is relatively large.

In the second specific example, at each processing time after the processing time t0, the lesion detection unit 34 compares the first score S1 with the first score threshold value Sth1 and compares the second score S2 with the second score threshold value Sth2 obtained at each processing time, respectively. Then, during the period from the processing time “t2” to the processing time “t3”, since the first score S1 exceeds the first score threshold value Sth1, the over-threshold consecutive count M increases. On the other hand, the first score S1 becomes equal to or smaller than the second threshold value Sth1 after the processing time t3, while the over-threshold consecutive count M does not exceed the threshold count Mth that is the initial value. Therefore, the lesion detection unit 34 determines that there is no lesion part in the above period.

On the other hand, at the processing time “t4”, the lesion detection unit 34 determines that the second score S2 is larger than the second score threshold value Sth2, and therefore sets the threshold count Mth to a predetermined relaxed value (i.e., a value relaxed form the initial value in terms of the condition for determining that there is a lesion part) which is smaller than the initial value. For example, the initial value of the threshold count Mth and the relaxed value of the threshold count Mth are previously stored in the memory 12 and the like, respectively.

Thereafter, after the processing time “t5”, the over-threshold consecutive count M increases since the first score S1 exceeds the first score threshold value Sth1. Then, during the period from the processing time t5 to the processing time “t6”, the first score S1 exceeds the first score threshold value Sth1 while the over-threshold consecutive count M becomes larger than the relaxed value of the threshold count Mth. Thus, the lesion detection unit 34 determines that there is a lesion part in the period from the processing time t5 to the processing time t0.

Thus, in such a situation in which the lesion detection accuracy based on the second model becomes higher than the lesion detection accuracy based on the first model, the second score S2 based on the second model reaches the second score threshold value Sth2, thereby suitably relaxing the condition for determining that there is a lesion part. Therefore, even in such a situation, the lesion detection unit 34 can accurately perform the lesion detection based on the first model. Besides, when there is a lesion which is easy to discriminate, the relaxation of the above-mentioned condition facilitates rapid detection of a lesion part by using a fewer number of endoscopic images Ia. In this case, since the number of endoscopic images Ia required for detection of a lesion is reduced, the possibility of initialization of the over-threshold consecutive count M due to instantaneous noises could be reduced.

Here, we supplementary description will be given of the advantage and disadvantage if either one of the first model based on a convolutional neural network and the second model based on SPRT for the lesion detection were used independently.

If a model based on a convolutional neural network is used for lesion detection, the presence or absence of detected lesion is determined by comparing the over-threshold consecutive count M with the threshold count Mth in order to improve the specificity. In such a lesion detection, there is such an advantage that it is possible to detect a lesion even under the circumstances in which logarithmic likelihood ratio to be calculated by the second model based on SPRT does not easily increase, e.g., there is no substantial variation in endoscopic images Ia with time. On the other hand, the lesion detection is susceptible to noises (including blurring) compared to the lesion detection based on the second model and it could need a lot of the number of endoscopic images Ia to detect a lesion part even when the lesion part is easily distinguishable. In contrast, the second model based on SPRT is robust to instantaneous noises and can promptly detect a lesion part that is easily distinguishable. Unfortunately, in such a case that there is little variation in endoscopic images Ia with time, the logarithmic likelihood ratio becomes hard to increase, and therefore the number of endoscopic image Ia required to detect a lesion could increase. Accordingly, in this example embodiment, by using them in combination, such a lesion detection having both advantages is suitably performed.

### (1-6) Processing Flow

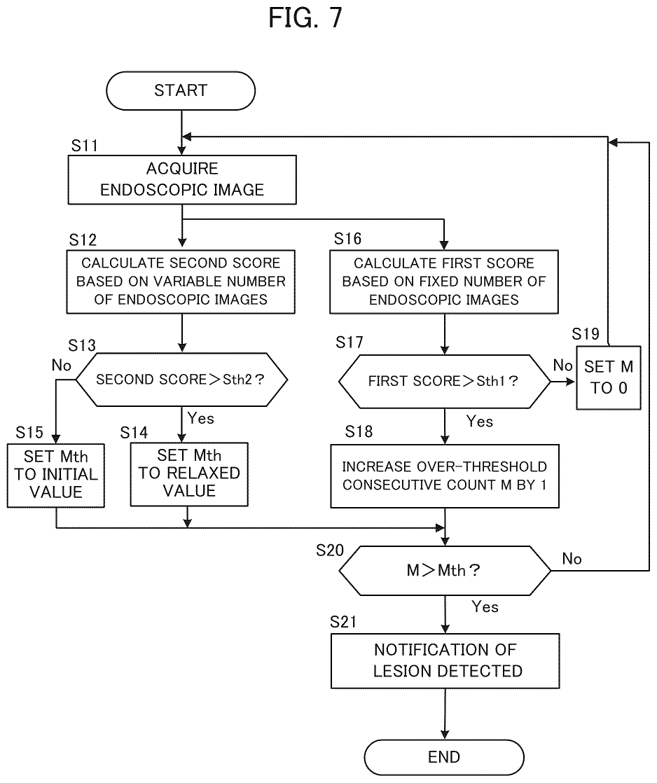

FIG. 7 is an example of a flowchart that is executed by the image processing device 1 according to the first example embodiment. The image processing device 1 repeatedly executes processing of the flowchart until the end of the endoscopic examination. For example, when the image processing device 1 detects a predetermined input or the like from the input unit 14 or the operation unit 36, it is determined that the endoscopic examination is completed.

First, the endoscopic image acquisition unit 30 of the image processing device 1 acquires an endoscopic image Ia (step S11). In this instance, the endoscopic image acquisition unit 30 of the image processing device 1 receives the endoscopic image Ia from the endoscope 3 through the interface 13. The display control unit 35 executes a process of displaying the endoscopic Ia acquired at step S11 on the display device 2. In addition, the feature extraction unit 31 generates feature data indicating the feature values of the acquired endoscopic image Ia.

Next, the second score calculation unit 33 calculates the second score S2 based on a variable number of endoscopic images Ia (step S12). In this case, for example, the second score calculation unit 33 calculates the second score S2 based on the feature data of the variable number of the endoscopic images Ia acquired at the current processing time and the past processing time(s) and the second model configured with reference to the second model information storage unit D2. In addition, the first score calculation unit 32 calculates the first score S1 based on a predetermined number of endoscopic images Ia in parallel with the process at step S12 (step S16). In this case, for example, the first score calculation unit 32 calculates the first score S1 based on the feature data of the predetermined number of the endoscopic images Ia acquired at the current processing time (and the past processing times) and the first model configured with reference to the first model information storage unit D1.

After executing the process at step S12, the lesion detection unit 34 determines whether or not the second score S2 is larger than the second score threshold value Sth2 (step S13). Then, if the second score S2 is larger than the second score threshold value Sth2 (step S13; Yes), the lesion detection unit 34 sets the threshold count Mth to a relaxed value smaller than the initial value (step S14). On the other hand, if the second score S2 is equal to or less than the second score threshold value Sth2 (step S13; No), the lesion detection unit 34 sets the threshold count Mth to the initial value (step S15).

Further, after executing the process at step S16, the lesion detection unit 34 determines whether or not the first score S1 is larger than the first score threshold value Sth1 (step S17). Then, if the first score S1 is larger than the first score threshold value Sth1 (step S17; Yes), the lesion detection unit 34 increases the over-threshold consecutive count M by 1 (step S18). It is herein assumed that the initial value of the over-threshold consecutive count M is set to 0. On the other hand, if the first score S1 is equal to or less than the first score threshold value Sth1 (step S17; No), the lesion detection unit 34 sets the over-threshold consecutive count M to 0 which is the initial value (step S19).

Next, after executing the process at step S14 or step S15, or after executing the process at step S18, the lesion detection unit 34 determines whether or not the over-threshold consecutive count M is larger than the threshold count Mth (step S20). Then, if the over-threshold consecutive count M is larger than the threshold count Mth (step S20; Yes), the lesion detection unit 34 determines that there is a lesion part, and then notifies the user that a lesion part is detected, by means of at least one of display and/or audio output (step S21). On the other hand, if the over-threshold consecutive count M is equal to or less than the threshold count Mth (step S20; No), the process returns to step S11.

### (1-7) Modifications

Next, a description will be given of modifications of the first example embodiment described above. The following modifications may be arbitrarily combined.

### (Modification 1-1)

The lesion detection unit 34 switched the threshold count Mth from the initial value to the relaxed value when the second score S2 exceeded the second score threshold value Sth2. On the other hand, instead of this mode, the lesion detection unit 34 may decrease, continuously or in stages, the threshold count Mth (i.e., may relax a condition for determining that there is a lesion part) with increase in the second score S2.

In this case, for example, correspondence information such as an expression or a look-up table indicating the relation between each possible second score S2 and the threshold count Mth suitable for the each possible second score S2 is stored in advance in the memory 12 or the like. The lesion detection unit 34 determines the threshold count Mth, based on the second score S2 and the above-described correspondence information. Even according to this mode, the lesion detection unit 34 sets the threshold count Mth in accordance with the second score S2, which enables the lesion detection unit 34 to detect a lesion while utilizing the advantages of both the first model and the second model.

Instead of changing the threshold count Mth based on the second score S2, or, in addition to this, the lesion detection unit 34 may change the first score threshold value Sth1 based on the second score S2. In this instance, for example, the lesion detection unit 34 may decrease the first score threshold value Sth1 in stages or continuously with increase in the second score S2. According to this mode, in a situation where the lesion detection based on the second model is effective, the lesion detection unit 34 can suitably relax the condition for detecting a lesion based on the first model and accurately perform the lesion detection.

### (Modification 1-3)

If it is determined that a predetermined condition based on the first score S1 is satisfied, the image processing device 1 may start the process of calculating the second score S2 and changing the threshold count Mth.

For example, the image processing device 1 does not perform calculation of the second score S2 by the second score calculation unit 33 after the start of the lesion detection process, and when it is determined that the first score S1 exceeds the first score threshold value Sth1, it starts calculating the second score S2 by the second score calculation unit 33, and changes the threshold count Mth (or the first score threshold value Sth1) in accordance with the second score S2 in the same manner as in the above-described example embodiment. On the other hand, after the calculation of the second score S2 by the second score calculation unit 33 is started, the image processing device 1 stops the calculation of the second score S2 by the second score calculation unit 33 again when it is determined that the first score S1 is equal to or less than the first score threshold value Sth1. The above-mentioned “predetermined condition” is not limited to the condition that the first score S1 is larger than the first score threshold value Sth1, and it may be any condition in which the probability that there is a lesion part is determined to become sufficiently high. Examples of such conditions include the condition that the first score S1 is larger than a predetermined threshold value that is smaller than the first score threshold value Sth1, the condition that the increment per unit time of the first score S1 (i.e., the derivative of the first score S1) is equal to or larger than a predetermined value, and the condition that the over-threshold consecutive count M is equal to or larger than a predetermined value.

Further, if the predetermined condition is satisfied and the calculation of the second score S2 is started, the image processing device 1 may calculate the second score S2 back to past processing time(s) and change the threshold count Mth (or the first score threshold value Sth1) based on the second score S2 at the past processing time. In this instance, for example, the image processing device 1 stores the feature data calculated by the feature extraction unit 31 at the past processing time in the memory 12 or the like, the second score calculation unit 33 calculates the second score S2 at the past processing time based on the feature data, and changes the threshold count Mth (or the first score threshold value Sth1) based on the second score S2.

According to this modification, the image processing device 1 limits the time period for calculating the second score S2 and can suitably reduce the computational burden.

### (Modification 1-4)

After the examination, the image processing device 1 may process a moving image configured by endoscopic images Ia generated during the endoscopic examination.

For example, if a moving image to be processed is designated based on the user input by the input unit 14 at any timing after the examination, the image processing device 1 repeatedly performs process of the flowchart shown in FIG. 7 for the time series endoscopic images Ia constituting the designated moving image until it is determined that the moving image has ended.

### Second Example Embodiment

### (2-1) Outline

In the second example embodiment, while detecting a lesion with reference to the second score S2 based on the second model, the image processing device 1 changes the second score threshold S2 to be compared with the second score S2 on the basis of the first score S1 that is based on the first model. Thus, in both of the situation in which the first model is easy to accurately detect the lesion part and the situation in which the second model is easy to accurately detect the lesion part, the image processing device 1 accurately detects the lesion part.

Hereinafter, substantially the same components of the endoscopic examination system 100 as in the first example embodiment will be denoted by the same reference numerals as appropriate and a description thereof will be omitted. The hardware configuration of the image processing device 1 according to the second example embodiment is substantially the same as the hardware configuration of the image processing device 1 shown in FIG. 2, and the functional block configuration of the processor 11 of the image processing device 1 according to the second example embodiment is substantially the same as the functional block configuration shown in FIG. 3.

In the second example embodiment, in the period in which the over-threshold consecutive count M increases, the lesion detection unit 34 decreases in stages or continuously the second score threshold value Sth2 (i.e., it relaxes the condition for determining that a lesion part is detected) with increase in the over-threshold consecutive count M. Thus, even in situations where the lesion detection based on the first model is effective, the lesion detection unit 34 suitably relaxes the condition for detecting a lesion based on the second model and therefore accurately executes the lesion detection.

In the second example embodiment, the second model is an example of the “selection model”, and the first model is an example of the “non-selection model”. In addition, the second score threshold value Sth2 is an example of the “parameter to be used for detection of a lesion based on a selection model”.

### (2-2) Concrete Example

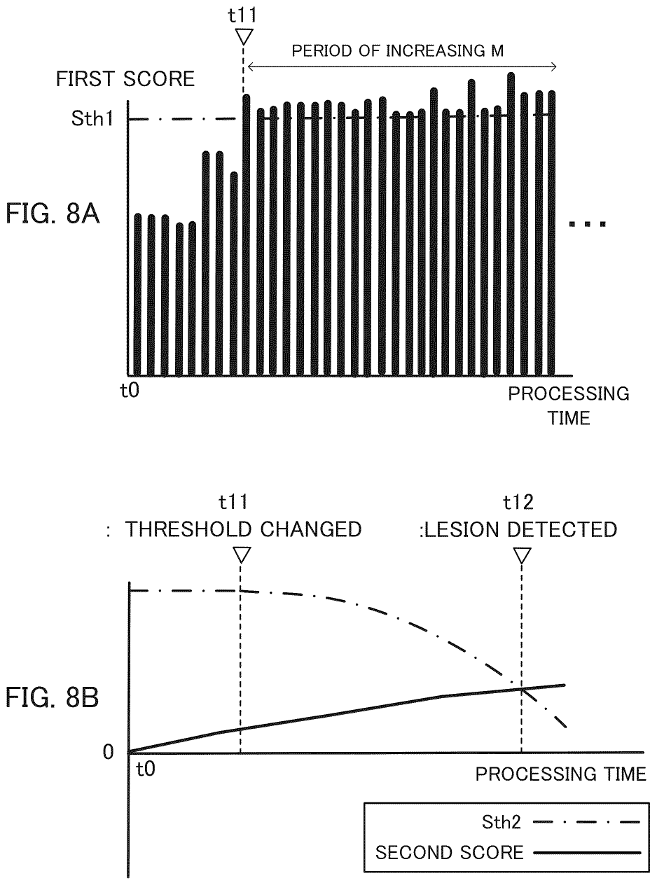

FIG. 8A is a graph showing the transition of the first score S1 from the processing time to at which the acquisition of the endoscope image Ia was started in the second example embodiment, and FIG. 8B is a graph showing the transition of the second score S2 from the processing time t0 in the second example embodiment. The specific example shown in FIG. 8A and FIG. 8B is an example of a lesion detection process in a situation where the accuracy of the lesion detection based on the first model becomes higher than the accuracy of the lesion detection based on the second model.

In this instance, at each processing time after the processing time t0, the lesion detection unit 34 compares the first score S1 obtained at each processing time with the first score threshold value Sth1, and compares the second score S2 obtained at each processing time with the second score threshold value Sth2. Then, the lesion detection unit 34 determines at the processing time “t11” that the first score S1 exceeds the first score threshold value Sth1 and then increases the over-threshold consecutive count M.

Then, after the processing time t11 that is the starting time of the period in which the over-threshold consecutive count M has increased, the lesion detection unit 34 changes the second score threshold value Sth2 in accordance with the over-threshold consecutive count M. Here, the lesion detection unit 34 continuously decreases the second score threshold value Sth2 with increase in the over-threshold consecutive count M. Then, at the processing time “t12” included in the period in which the over-threshold consecutive count M has increased, since the second score S2 is larger than the second score threshold value Sth2, the lesion detection unit 34 determines, at the processing time t12, that there is a lesion part.

Thus, even in a situation in which the accuracy of the detection of a lesion based on the first model becomes higher than the accuracy of the detection of a lesion based on the second model, the lesion detection unit 34 decreases the second score threshold value Sth2 with an increase in the over-threshold consecutive count M, thereby relaxing the condition for detecting a lesion related to the second score S2 based on the second model to accurately perform the lesion detection. Further, even in a situation where the accuracy of the detection of a lesion based on the second model is higher than the accuracy of the detection of a lesion based on the first model, since the second score Sth2 based on the second model reaches the second score threshold value Sth2 even if the second score threshold S2 does not change, the lesion detection unit 34 can accurately detect a lesion.

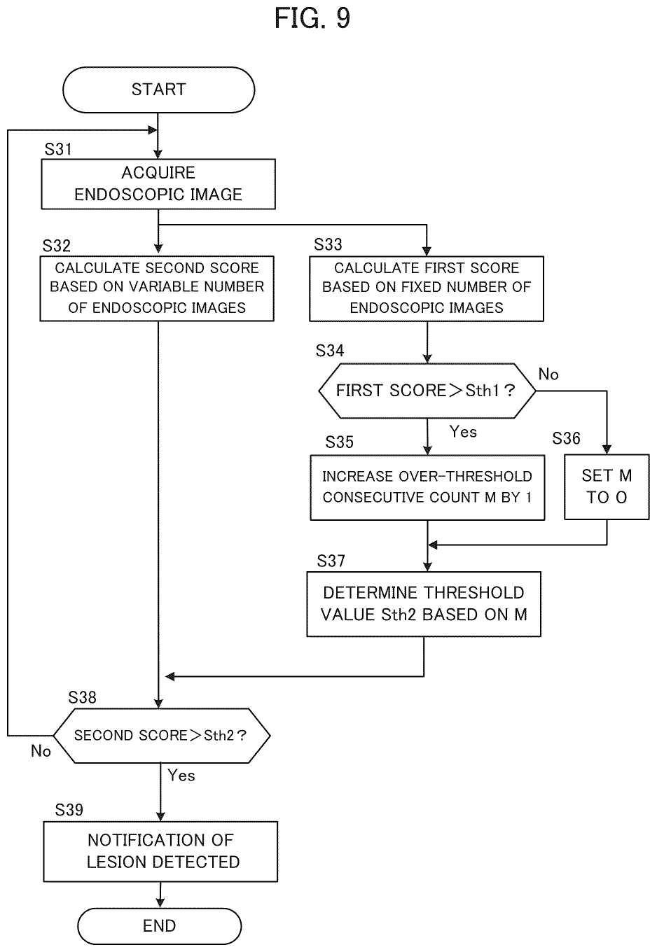

FIG. 9 is an example of a flowchart that is executed by the image processing device 1 in the second example embodiment. The image processing device 1 repeatedly executes processing of the flowchart until the end of the endoscopic examination.

First, the endoscopic image acquisition unit 30 of the image processing device 1 acquires an endoscopic image Ia (step S31). In this instance, the endoscopic image acquisition unit 30 of the image processing device 1 receives the endoscopic image Ia from the endoscope 3 through the interface 13. The display control unit 35 executes a process of displaying the endoscopic Ia acquired at step S31 on the display device 2. In addition, the feature extraction unit 31 generates feature data indicating the feature values of the acquired endoscopic image Ia.

Next, the second score calculation unit 33 calculates the second score S2 based on a variable number of endoscopic images Ia (step S32). In this case, for example, the second score calculation unit 33 calculates the second score S2 based on the feature data of the variable number of the endoscopic images Ia acquired at the current processing time and the past processing time(s) and the second model configured with reference to the second model information storage unit D2. In addition, the first score calculation unit 32 calculates the first score S1 based on a predetermined number of endoscopic images Ia in parallel with the process at step S32 (step S33). In this case, for example, the first score calculation unit 32 calculates the first score S1 based on the feature data of the predetermined number of the endoscopic images Ia acquired at the current processing time (and the past processing times) and the first model configured with reference to the first model information storage unit D1.

After executing the process at step S33, the lesion detection unit 34 determines whether or not the first score S1 is larger than the first score threshold value Sth1 (step S34). Then, if the first score S1 is larger than the first score threshold value Sth1 (step S34; Yes), the lesion detection unit 34 increases the over-threshold consecutive count M by 1 (step S35). It is herein assumed that the initial value of the over-threshold consecutive count M is set to 0. On the other hand, if the first score S1 is equal to or smaller than the first score threshold value Sth1 (step S34; No), the lesion detection unit 34 sets the over-threshold consecutive count M to 0 which is the initial value (step S36).

Then, after executing the process at step S35 or step S36, based on the over-threshold consecutive count M, the lesion detection unit 34 determines the second score threshold value Sth2 that is a threshold value to be compared with the second score S2 (step S37). In this instance, for example, the lesion detection unit 34 refers to a previously-stored expression or look-up table or the like, and decreases the second score threshold value Sth2 with increase in the over-threshold consecutive count M.

Then, after executing the processes at step S32 and step S37, the lesion detection unit 34 determines whether the second score S2 is larger than the second score threshold value Sth2 (step S38). Then, if the second score S2 is larger than the second score threshold value Sth2 (step S38; Yes), the lesion detection unit 34 determines that there is a lesion part, and therefore outputs the notification indicating that a lesion part is detected by at least one of the display and/or sound output (step S39). On the other hand, if the second score S2 is equal to or less than the second score threshold value Sth2 (step S38; No), it gets back to the process at step S31.

### (2-4) Modifications

Next, a description will be given of modifications of the second example embodiment described above. The following modifications may be arbitrarily combined.

### (Modification 2-1)

The image processing device 1 may start the process of calculating the first score S1 and changing the second score threshold value Sth2 by the first model when it is determined that a predetermined condition based on the second score S2 is satisfied.

For example, after the start of the lesion detection process, the image processing device 1 does not perform calculation of the first score S1 by the first score calculation unit 32 and starts calculating the first score S1 by the first score calculation unit 32 if the second score S2 is larger than a predetermined threshold value (e.g., 0) smaller than the second score threshold value Sth2. Then, the image processing device 1 changes the second score threshold value Sth2 in accordance with the over-threshold consecutive count M in the same manner as in the above-described example embodiment. On the other hand, after the calculation of the first score S1 by the first score calculation unit 32 is started, the image processing device 1 stops the calculation of the first score S1 by the first score calculation unit 32 again if it is determined that the second score S2 is equal to or less than the predetermined threshold. It is noted that the above-mentioned “predetermined condition” is not limited to the condition that the second score S2 is larger than the predetermined threshold, and it may be any condition in which the probability that there is a lesion part is determined to become sufficiently high. Examples of such conditions include the condition that the increment per unit time of the second score S2 (i.e., the derivative of the second score S2) is equal to or larger than a predetermined value.

In addition, if the predetermined condition is satisfied and therefore the calculation of the first score S1 is started, the image processing device 1 may calculate the first score S1 back to the past processing time(s) and change the second score threshold value Sth2 based on the first score S1 at the past processing time. In this instance, for example, the image processing device 1 stores the feature data calculated by the feature extraction unit 31 at the past processing times in the memory 12 or the like, and the first score calculation unit 32 calculates the first score S1 at the past processing time based on the feature data, and change the second score threshold value Sth2 at the past processing time based on the first score S1. In this instance, the image processing device 1 compares the second score S2 with the second score threshold value Sth2 at every past processing time t0 determine whether or not there is a lesion part.

According to this modification, the image processing device 1 limits the time period for calculating the second score S2 and can suitably reduce the computational burden.

### (Modification 2-2)

After the examination, the image processing device 1 may process a moving image configured by endoscopic images Ia generated during the endoscopic examination.

For example, if the moving image to be processed is designated based on the user input by the input unit 14 at any timing after the examination, the image processing device 1 repeatedly performs processing of the flowchart shown in FIG. 9 for time-series endoscopic images Ia constituting the image until it is determined that the target moving image has ended.

### Third Example Embodiment

In the third example embodiment, the image processing device 1 switches between the lesion detection process based on the first example embodiment and the lesion detection process based on the second example embodiment, based on the degree of variation between time series endoscopic images Ia. Hereafter, the lesion detection process based on the first example embodiment is referred to as “first model based lesion detection process”, and the lesion detection process based on the second example embodiment is referred to as “second model based lesion detection process”.

Hereinafter, substantially the same components of the endoscopic examination system 100 as in the first example embodiment will be denoted by the same reference numerals as appropriate and a description thereof will be omitted. The hardware configuration of the image processing device 1 according to the third example embodiment is the same as the hardware configuration of the image processing device 1 shown in FIG. 2, and the functional block configuration of the processor 11 of the image processing device 1 according to the third example embodiment is the same as the functional block configuration shown in FIG. 3.

In the third example embodiment, the lesion detection unit 34 calculates a score (also referred to as “variation score”) representing the degree of variation between the endoscopic image Ia (also referred to as a “current processing image”) at the time index t representing the current processing time and the endoscopic image Ia (also referred to as “past image”) acquired at the time (i.e., the time index “t−1”) immediately before the current processing time. The variation score increases with increase in the degree of variation between the current processing image and the past image. For example, the lesion detection unit 34 calculates, as the variation score, a value of any similarity index based on comparison of images (i.e., comparison between images). Examples of the similarity index in this case include the correlation coefficient, SSIM (Structural Similarity) index, PSNR (Peak Signal-to-Noise Ratio) index, and the square error between corresponding pixels. Instead of calculating the variation score by directly comparing the current processing image with the past image, the lesion detection unit 34 may compare the feature values of the current processing image with the feature values of the past image to thereby calculate the degree of similarity as the variation score.

Then, if the variation score is equal to or smaller than a predetermined threshold value (also referred to as “variation threshold value”), the lesion detection unit 34 performs the first model based lesion detection process. In other words, in this case, the lesion detection unit 34 determines the threshold count Mth based on the second score S2 while determining that there is a lesion part if the over-threshold consecutive count M based on the first score S1 becomes larger than the threshold count Mth. For example, the variation threshold value is stored in advance in the memory 12 or the like. On the other hand, if the variation score is larger than the variation threshold value, the lesion detection unit 34 performs the second model based lesion detection process. That is, in this case, the lesion detection unit 34 determines the second score threshold value Sth2 based on the first score S1 while determining that there is a lesion part if the second score S2 becomes larger than the second score threshold value Sth2. As described above, in the third example embodiment, the lesion detection unit 34 selects the selected model, which is a model to be used for lesion detection, from the first model and the second model based on the degree of variation in the endoscopic images Ia.

Here, a supplementary description will be given of the effect according to the third example embodiment. As described in the first example embodiment, the lesion detection based on the first model is advantageous in that the first model is capable of detecting a lesion even under conditions where the logarithmic likelihood ratio based on the second model does not easily increase when there is no temporal change in the endoscopic images Ia (i.e., when the variation score is relatively low), whereas the lesion detection based on the second model is advantageous in that the second model is robust to instantaneous noises and capable of promptly detecting a lesion part that is easily distinguishable. In view of the above, in the third example embodiment, if the variation score is equal to or less than the variation threshold and therefore the lesion detection based on the first model is effective, the lesion detection unit 34 determines whether or not there is a lesion part on the basis of the first score S1 and the over-threshold consecutive count M. If the variation score exceeds the variation threshold and therefore the lesion detection based on the second model is effective, the lesion detection unit 34 determines whether or not there is a lesion part on the basis of the second score S2. Thus, it is possible to suitably increase the lesion detection accuracy.

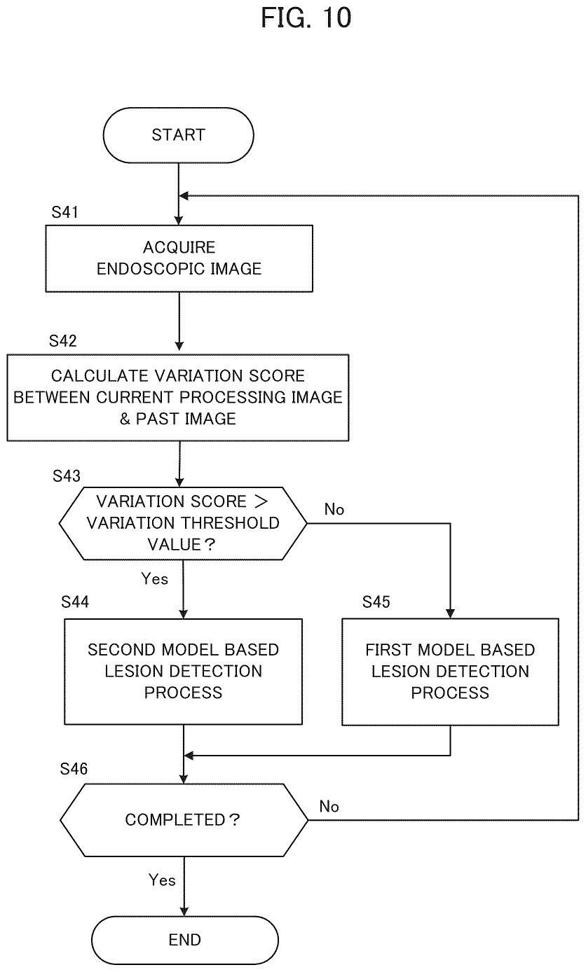

FIG. 10 is an example of a flowchart that is executed by the image processing device 1 in the third example embodiment.

First, the endoscopic image acquisition unit 30 of the image processing device 1 acquires the endoscopic image Ia (step S41). In this instance, the endoscopic image acquisition unit 30 of the image processing device 1 receives the endoscopic image Ia from the endoscope 3 through the interface 13. The display control unit 35 executes a process of displaying the endoscopic image Ia acquired at step S41 on the display device 2.

Next, the lesion detection unit 34 calculates the variation score based on the current processing image which is the endoscopic image Ia obtained at step S41 at the current processing time and the past image which is the endoscopic image Ia obtained at step S42 at the immediately preceding processing time. Then, the lesion detection unit 34 determines whether or not the variation score is larger than the variation threshold value (step S43). Then, if the variation score is larger than the variation threshold value (step S43; Yes), the image processing device 1 performs the second model based lesion detection process (step S44). In this instance, the image processing device 1 executes the flowchart shown in FIG. 9 excluding the process at step S31 which overlaps with the process at step S41. If it is determined at step S38 that the second score S2 is equal to or less than the second score threshold value Sth2, it proceeds with the process at step S46. On the other hand, if the variation score is equal to or less than the variation threshold value (step S43; No), the image processing device 1 executes the first model based lesion detection process (step S45). In this instance, the image processing device 1 executes the flowchart shown in FIG. 7 excluding the process at step S11 which overlaps with the process at step S41. In contrast, if it is determined at step S20 that the over-threshold consecutive count M is equal to or less than the threshold count Mth or if the process at step S19 is done, it proceeds with the process at step S46.

Then, the image processing device 1 determines whether or not the endoscopic examination is completed (step S46). For example, the image processing device 1 determines that the endoscopic examination has been completed if a predetermined input or the like to the input unit 14 or the operation unit 36 is detected. If it is determined that the endoscopic examination has been completed (step S46; Yes), the image processing device 1 ends the process of the flowchart. On the other hand, if it is determined that the endoscopic examination has not been completed (step S46; No), the image processing device 1 gets back to the process at step S41.

### Fourth Example Embodiment

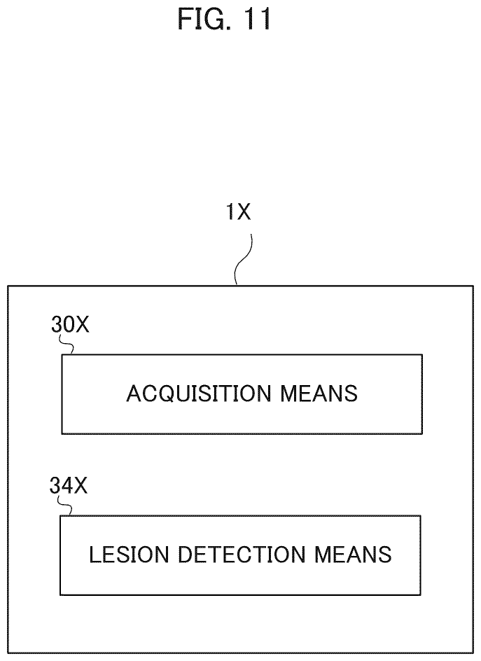

FIG. 11 is a block diagram of an image processing device 1X according to a fourth example embodiment. The image processing device 1X includes an acquisition means 30X and a lesion detection means 34X. The image processing device 1X may be configured by a plurality of devices.

The acquisition means 30X is configured to acquire an endoscopic image obtained by photographing an examination target by a photographing unit provided in an endoscope. In this instance, the acquisition means 30X may immediately acquire the endoscopic image generated by the photographing unit, or may acquire, at a predetermined timing, the endoscopic image previously generated by the photographing unit and stored in the storage device. Examples of the acquisition means 30X include the endoscopic image acquisition unit 30 in the first example embodiment to the third example embodiment.

The lesion detection means 34X is configured to detect a lesion based on a selection model which is selected from a first model and a second model, the first model being configured to make an inference regarding a lesion of the examination target based on a predetermined number of endoscopic images, the second model being configured to make an inference regarding a lesion of the examination target based on a variable number of endoscopic images. In addition, the lesion detection means 34X is configured to change a parameter to be used for detection of the lesion based on a non-selection model that is the first model or the second model other than the selection model. Examples of the “selection model” include the “first model” in the first example embodiment, the “first model” in the first model based lesion detection process in the third example embodiment, the “second model” in the second example embodiment, the “second model” in the second model based lesion detection process in the third example embodiment. Examples of the “parameter to be used for detection of the lesion based on the selection model” include the “threshold count Mth” and the “first score threshold value Sth1” in the first example embodiment, “threshold count Mth” and the “first score threshold value Sth1” in the first model based lesion detection process in the third example embodiment, the “second score threshold value Sth2” in the second example embodiment, and the “second score threshold value Sth2” in the second model based lesion detection process in the third example embodiment. It is noted that selection of the “selection model” and “non-selected model” herein is not limited to the case that is made autonomously based on the variation score as in the third example embodiment and it may be determined in advance by setting as in the first example embodiment or the second example embodiment. Examples of the lesion detection means 34X include the first score calculation unit 32, the second score calculation unit 33, and the lesion detection unit 34 in the first example embodiment to the third example embodiment.

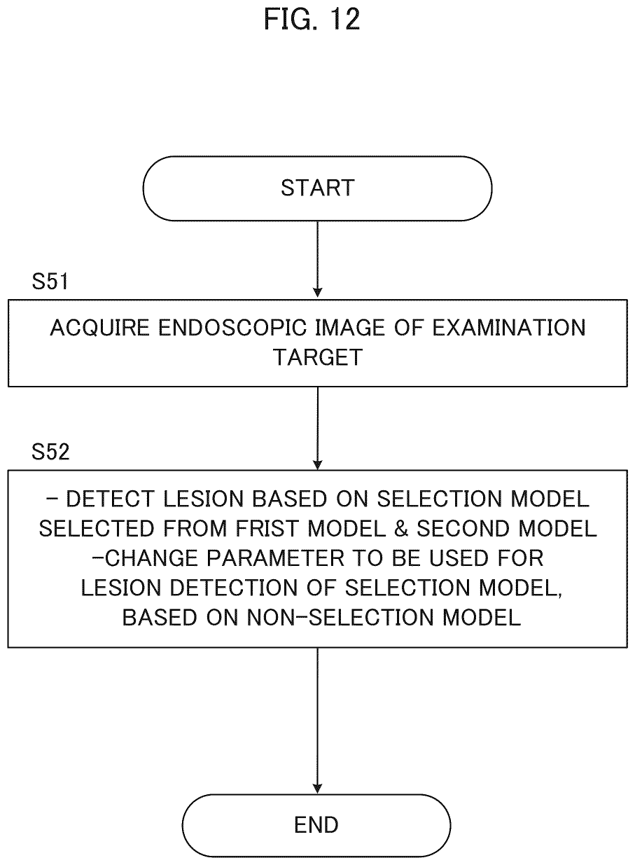

FIG. 12 is an example of a flowchart showing a processing procedure in the fourth example embodiment. First, the acquisition means 30X is configured to acquire an endoscopic image obtained by photographing an examination target by a photographing unit provided in an endoscope (step S51). The lesion detection means 34X detects a lesion based on a selection model which is selected from a first model and a second model, the first model being configured to make an inference regarding a lesion of the examination target based on a predetermined number of endoscopic images, the second model being configured to make an inference regarding a lesion of the examination target based on a variable number of endoscopic images. In addition, the lesion detection means 34X changes a parameter to be used for detection of the lesion based on a non-selection model that is the first model or the second model other than the selection model (step S52).

According to the fourth example embodiment, the image processing device 1X can accurately detect the lesion part included in the endoscopic image.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### [Supplementary Note 1]

An image processing device comprising:
an acquisition means configured to acquire an endoscopic image obtained by photographing an examination target by a photographing unit provided in an endoscope; and
a lesion detection means configured to detect a lesion based on a selection model which is selected from a first model and a second model,
   the first model being configured to make an inference regarding a lesion of the examination target based on a predetermined number of endoscopic images,
   the second model being configured to make an inference regarding a lesion of the examination target based on a variable number of endoscopic images,
wherein the lesion detection means is configured to change a parameter to be used for detection of the lesion based on a non-selection model that is the first model or the second model other than the selection model.

### [Supplementary Note 2]

The image processing device according to Supplementary Note 1,
wherein the parameter is a parameter defining a condition for determining that the lesion is detected, and
wherein the lesion detection means is configured to change the parameter so that, the higher a degree of confidence of presence of the lesion indicated by a score calculated by the non-selection model is, the more the condition is relaxed.

### [Supplementary Note 3]

The image processing device according to Supplementary Note 1,
wherein the first model is a deep learning model whose architecture includes a convolutional neural network.

### [Supplementary Note 4]

The image processing device according to Supplementary Note 1,
wherein the selection model is the first model,
wherein the lesion detection means is configured to determine that the lesion is detected if a consecutive number of times a degree of confidence of presence of the lesion exceeds a predetermined threshold value is larger than a predetermined number of times,
   the degree of confidence being indicated by a score calculated by the first model from the endoscopic images acquired in time series,
wherein the parameter is at least one of the predetermined number of times and/or the predetermined threshold value, and
wherein the lesion detection means is configured to change at least one of the predetermined number of times and/or the predetermined threshold value, based on a score calculated by the second model.

### [Supplementary Note 5]

The image processing device according to Supplementary Note 1,
wherein the second model is a model based on SPRT.

### [Supplementary Note 6]

The image processing device according to Supplementary Note 1,
wherein the selection model is the second model,
wherein the lesion detection means is configured to determine that the lesion is detected if a degree of confidence of presence of the lesion exceeds a predetermined value,
   the degree of confidence being indicated by a score calculated by the second model,
wherein the parameter is the predetermined threshold value, and
wherein the lesion detection means is configured to change the predetermined threshold value based on a score calculated by the first model.

### [Supplementary Note 7]

The image processing device according to Supplementary Note 1,
wherein the lesion detection means is configured to determine the selection model from the first model and the second model, based on a degree of variation between the endoscopic images.

### [Supplementary Note 8]

The image processing device according to Supplementary Note 1,
wherein the lesion detection means is configured to start calculating a score based on the non-selection model if it is determined that a predetermined condition based on a score calculated by the selection model is satisfied.

### [Supplementary Note 9]

The image processing device according to Supplementary Note 1, further comprising
an output control means configured to display or output, by audio, information regarding a detection result of the lesion by the lesion detection means.

### [Supplementary Note 10]

The image processing device according to Supplementary Note 9,
wherein the output control means is configured to output the information regarding the detection result of the lesion and information regarding the selection model to assist in decision making by an examiner.

### [Supplementary Note 11]

An image processing method executed by a computer, the image processing method comprising:
acquiring an endoscopic image obtained by photographing an examination target by a photographing unit provided in an endoscope;
detecting a lesion based on a selection model which is selected from a first model and a second model,
   the first model being configured to make an inference regarding a lesion of the examination target based on a predetermined number of endoscopic images,
   the second model being configured to make an inference regarding a lesion of the examination target based on a variable number of endoscopic images; and
changing a parameter to be used for detection of the lesion based on a non-selection model that is the first model or the second model other than the selection model.

### [Supplementary Note 12]

A storage medium storing a program executed by a computer, the program causing the computer to:
acquire an endoscopic image obtained by photographing an examination target by a photographing unit provided in an endoscope;
detect a lesion based on a selection model which is selected from a first model and a second model,
   the first model being configured to make an inference regarding a lesion of the examination target based on a predetermined number of endoscopic images,
   the second model being configured to make an inference regarding a lesion of the examination target based on a variable number of endoscopic images; and
change a parameter to be used for detection of the lesion based on a non-selection model that is the first model or the second model other than the selection model.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1X Image Processing Device
2 Display device
3 Endoscope
11 Processor
12 Memory
13 Interface
14 Input unit
15 Light source unit
16 Audio output unit
100 Endoscopic examination system

What is claimed is:

1. An image processing device comprising:
at least one memory configured to store instructions; and
at least one processor configured to execute the instructions to:
acquire time-series images photographed by a camera of an endoscope;
detect a lesion based on a selection model which is selected from a first model and a second model,
the first model being configured to make an inference regarding a lesion based on a predetermined number of images from the time-series images,
the second model being configured to make an inference regarding a lesion based on a variable number of images from the time-series images;
display a first score transition graph and a second score transition graph, the first score transition graph indicating a transition of first score calculated by the first model from the images, the second score transition graph indicating a transition of second score calculated by the second model from the images; and
change a parameter to be used for detection of the lesion based on a non-selection model that is the first model or the second model other than the selection model.

2. The image processing device according to claim 1,
wherein the at least one processor is configured to execute the instructions to display a line indicating a criterion value for determining for determining the presence or absence of a lesion.

3. The image processing device according to claim 1,
wherein the at least one processor is configured to display a real time image of the image and a result of detecting the lesion.

4. The image processing device according to claim 1,
wherein the parameter is a parameter defining a condition for determining that the lesion is detected, and
wherein the at least one processor is configured to execute the instructions to change the parameter so that, the higher a degree of confidence of presence of the lesion indicated by a score calculated by the non-selection model is, the more the condition is relaxed.

5. The image processing device according to claim 4,
wherein the parameter in a case where the selection model is the first model is at least one of the predetermined number of times and the predetermined threshold value, and
wherein the at least one processor is configured to, in a case where the selection model is the first model, change at least one of the predetermined number of times and the predetermined threshold value, based on the score calculated by the second model.

6. The image processing device according to claim 1,
wherein the first model is a deep learning model whose architecture includes a convolutional neural network.

7. The image processing device according to claim 1,
wherein the selection model is the first model,
wherein the at least one processor is configured to execute the instructions to determine that the lesion is detected if a consecutive number of times a degree of confidence of presence of the lesion exceeds a predetermined threshold value is larger than a predetermined number of times,
the degree of confidence being indicated by a score calculated by the first model from the images acquired in time series,
wherein the parameter is at least one of the predetermined number of times and/or the predetermined threshold value, and
wherein the at least one processor is configured to execute the instructions to change at least one of the predetermined number of times and/or the predetermined threshold value, based on a score calculated by the second model.

8. The image processing device according to claim 1,
wherein the second model is a model based on SPRT.

9. The image processing device according to claim 1,
wherein the selection model is the second model,
wherein the at least one processor is configured to execute the instructions to determine that the lesion is detected if a degree of confidence of presence of the lesion exceeds a predetermined value,
the degree of confidence being indicated by a score calculated by the second model,
wherein the parameter is the predetermined threshold value, and
wherein the at least one processor is configured to execute the instructions change the predetermined threshold value based on a score calculated by the first model.

10. The image processing device according to claim 1,
wherein the at least one processor is configured to execute the instructions to determine the selection model from the first model and the second model, based on a degree of variation between the images.

11. The image processing device according to claim 1,
wherein the at least one processor is configured to execute the instructions to start calculating a score based on the non-selection model if it is determined that a predetermined condition based on a score calculated by the selection model is satisfied.

12. The image processing device according to claim 1,
wherein the at least one processor is configured to further execute the instructions to display or output, by audio, information regarding a detection result of the lesion.

13. The image processing device according to claim 12,
wherein the at least one processor is configured to execute the instructions to output the information regarding the detection result of the lesion and information regarding the selection model to assist in decision making by an examiner.

14. An image processing method executed by a computer, the image processing method comprising:
acquiring time-series images photographed by a camera of an endoscope;
detecting a lesion based on a selection model which is selected from a first model and a second model,
the first model being configured to make an inference regarding a lesion based on a predetermined number of images from the time-series images,
the second model being configured to make an inference regarding a lesion based on a variable number of images from the time-series images;
displaying a first score transition graph and a second score transition graph, the first score transition graph indicating a transition of first score calculated by the first model from the images, the second score transition graph indicating a transition of second score calculated by the second model from the images; and
changing a parameter to be used for detection of the lesion based on a non-selection model that is the first model or the second model other than the selection model.

15. The image processing method according to claim 14, the image processing method comprising
display a line indicating a criterion value for determining for determining the presence or absence of a lesion.

16. The image processing method according to claim 14, the image processing method comprising
wherein the at least one processor is configured to display a real time image of the endoscopic image and a result of detecting the lesion.

17. The image processing method according to claim 14,
wherein the parameter is a parameter defining a condition for determining that the lesion is detected, and
the image processing method comprising change the parameter so that, the higher a degree of confidence of presence of the lesion indicated by a score calculated by the non-selection model is, the more the condition is relaxed.

18. The image processing method according to claim 17,
wherein the parameter in a case where the selection model is the first model is at least one of the predetermined number of times and the predetermined threshold value, and
the image processing method comprising, in a case where the selection model is the first model, change at least one of the predetermined number of times and the predetermined threshold value, based on the score calculated by the second model.

19. The image processing method according to claim 14,
wherein the first model is a deep learning model whose architecture includes a convolutional neural network.

20. A non-transitory computer readable storage medium storing a program executed by a computer, the program causing the computer to:
acquire time-series images photographed by a camera of an endoscope;
detect a lesion based on a selection model which is selected from a first model and a second model,
the first model being configured to make an inference regarding a lesion based on a predetermined number of images from the time-series images,
the second model being configured to make an inference regarding a lesion based on a variable number of images from the time-series images;
display a first score transition graph and a second score transition graph, the first score transition graph indicating a transition of first score calculated by the first model from the images, the second score transition graph indicating a transition of second score calculated by the second model from the images; and
change a parameter to be used for detection of the lesion based on a non-selection model that is the first model or the second model other than the selection model.
